(19) United States
(12) Patent Application Publication
(10) Pub. No.: US 2025/0114385 A1
(43) Pub. Date: Apr. 10, 2025

(54) ORGANIC GLUCOSE BEVERAGE AND GLUCOSE MONITORING SYSTEM FOR DIABETES SCREENING AND DIAGNOSIS

(71) Applicant: Angie Girard, San Rafael, CA (US)

(72) Inventor: Angie Girard, San Rafael, CA (US)

(21) Appl. No.: 18/907,709

(22) Filed: Oct. 7, 2024

Related U.S. Application Data
(60) Provisional application No. 63/588,005, filed on Oct. 5, 2023.

(51) Int. Cl. A61K 31/7004; A23L 2/02; A23L 2/52; A23L 2/68; A61B 5/00; A61K 9/00; A61K 47/12; A61K 49/00; G01N 33/66

(52) U.S. Cl. CPC A61K 31/7004; A23L 2/02; A23L 2/52; A23L 2/68; A61B 5/4866; A61K 9/0095; A61K 47/12; A61K 49/0004; G01N 33/66; A61B 2503/045; G01N 2800/042

(57) ABSTRACT

An organic glucose beverage and a novel glucose monitoring system are provided that significantly improves process of diabetes screening, diagnosis, and treatment of hypoglycemia including gestational diabetes mellitus (GDM), type-1 diabetes, type-2 diabetes, and hypoglycemia. The organic glucose beverage comprises organic glucose in form of dextrose monohydrate, juice concentrates, citric acid, and water. The organic glucose beverage is provided in the form of a ready-to-drink beverage. The glucose monitoring system comprises an organic glucose beverage, a device for blood sugar monitoring, and a device for the determination of a predefined threshold value. The present invention provides first time an organic beverage for gestational diabetes mellitus (GDM), type-1 diabetes, type-2 diabetes, and hypoglycemia. The organic glucose beverage of the present invention is healthier, well tolerated, and does not include artificial flavors, dyes, or toxic preservatives which sets it apart from conventional glucose solutions for use in diabetes screening and diagnosis.

### CROSS REFERENCE TO RELATED APPLICATION(S)

This application claims the priority to U.S. provisional patent application bearing No. 63/588,005 filed on Oct. 5, 2023 and titled “ORGANIC GLUCOSE BEVERAGE AND GLUCOSE MONITORING SYSTEM FOR DIABETES SCREENING AND DIAGNOSIS”

### TECHNICAL FIELD

The present invention relates to organic glucose beverage and monitoring system for diabetes screening and diagnosis. More particularly, the present invention relates to a novel organic glucose beverage and a glucose monitoring system to enhance and simplify a process of diabetes screening, diagnosis, and treatment of hypoglycemia including gestational diabetes mellitus (GDM), type-1 diabetes, type-2 diabetes, and human and veterinary treatment of hypoglycemia.

### BACKGROUND

Diabetes mellitus is a prominent global health concern, affecting individuals across various age groups and demographic profiles. Gestational diabetes mellitus (GDM) is a prevalent medical condition that affects pregnant women, characterized by elevated blood glucose levels during pregnancy. Accurate and efficient GDM diagnosis is essential for effective medical management and the prevention of associated complications for the well-being of both the mother and child.

However, traditional approaches to diabetes screening, diagnosis, and treatment of hypoglycemia often rely on glucose solutions containing synthetic additives, preservatives, and artificial dyes. While these glucose solutions serve their intended diagnostic purpose but are associated with potential health risks and operational challenges.

Conventional glucose solutions may contain synthetic dyes, artificial flavorings, and preservatives, raising concerns about patient safety, particularly in pregnant women and children. These additives have the potential to compromise a patient's well-being during a screening process.

Furthermore, some glucose solutions necessitate refrigeration, adding logistical complexities and costs to healthcare facilities. Additionally, powdered formulations that require manual mixing with water result in inconsistencies in dosage and concentration.

The taste and composition of traditional glucose solutions can be unpalatable to patients, particularly children, leading to patient resistance and non-compliance with the screening process. Such reluctance may compromise the accuracy of the diagnosis.

Additionally, production, storage, and disposal of glucose solutions containing synthetic ingredients contribute to healthcare costs, particularly in larger healthcare facilities and institutions.

Therefore, given these limitations and challenges, there is a pressing demand for a novel ready-to-drink organic glucose beverage for diabetes screening, diagnosis, and treatment of hypoglycemia including gestational diabetes mellitus (GDM), type-1 diabetes, type-2 diabetes, and human and veterinary treatment of hypoglycemia that mitigates shortcomings of conventional glucose screening methods offering a safer, more convenient, and cost-effective solution that enhances patient compliance and healthcare efficiency.

### SUMMARY

Embodiments of the present invention relate to an organic glucose beverage and a novel glucose monitoring system that significantly improves a process of diabetes screening, diagnosis, and treatment of hypoglycemia including gestational diabetes mellitus (GDM), type-1 diabetes, type-2 diabetes, and human and veterinary treatment of hypoglycemia.

In an embodiment of the present invention, an organic glucose beverage for diabetes screening, diagnosis, and treatment of hypoglycemia including gestational diabetes mellitus (GDM), type-1 diabetes, type-2 diabetes, and human and veterinary treatment of hypoglycemia is provided. The organic glucose beverage comprises 19-40% w/v organic glucose in the form of dextrose monohydrate, 3.5% w/v juice concentrates; 0.5% w/v citric acid, and balance to 100% w/v water. All ingredients are taken in a liquid form. The organic glucose beverage is provided in the form of a ready-to-drink beverage. The organic glucose beverage is both shelf-stable and free from artificial dyes, food colorings, and toxic preservatives.

In another embodiment of the present invention, a glucose monitoring system for diabetes screening, diagnosis, and treatment of hypoglycemia including gestational diabetes mellitus (GDM), type-1 diabetes, type-2 diabetes, and human and veterinary treatment of hypoglycemia is provided. The glucose monitoring system comprises a container holding an organic glucose beverage, one or more hardware processors, and a memory unit operatively connected to the one or more hardware processors, wherein the memory unit comprises a set of computer-readable instructions in form of a plurality of modules, configured to be executed by the one or more hardware processors. The plurality of modules comprises a measuring module for determining a blood sugar level in a sample obtained from a user, a comparator module configured for comparing the determined blood sugar level with a predefined blood sugar threshold value, a diagnostic module configured to analyze the blood sugar level determined by the measuring module. The glucose monitoring system is configured to administer the organic glucose beverage, measure blood sugar levels, and determine the presence of gestational diabetes mellitus (GDM), type-1 diabetes, and type-2 diabetes based on the measured blood sugar levels and the predefined threshold value.

The present invention provides first time an organic beverage for diabetes screening, diagnosis, and treatment of hypoglycemia including gestational diabetes mellitus (GDM), type-1 diabetes, type-2 diabetes, and human and veterinary treatment of hypoglycemia. The organic glucose beverage of the present invention is healthier, well tolerated, and does not include artificial flavors, dyes, or toxic preservatives which sets it apart for intended use from conventional glucose solutions for diabetes screening, diagnosis, and treatment of hypoglycemia including gestational diabetes mellitus (GDM), type-1 diabetes, type-2 diabetes, and human and veterinary treatment of hypoglycemia.

To further clarify the advantages and features of the present invention, a more particular description of the invention will follow by reference to specific embodiments thereof, which are illustrated in the appended figures. It is to be appreciated that these figures depict only typical embodiments of the invention and are therefore not to be considered limiting in scope. The invention will be described and explained with additional specificity and detail with the appended figures.

### BRIEF DESCRIPTION OF THE DRAWINGS

The disclosure will be described and explained with additional specificity and detail with the accompanying figures in which:

FIG. 1 illustrates an exemplary block diagram representation of a glucose monitoring system 102 for diabetes screening, diagnosis, and treatment of hypoglycemia including gestational diabetes mellitus (GDM), type-1 diabetes, type-2 diabetes, and human and veterinary treatment of hypoglycemia, in accordance with an embodiment of the present disclosure;

FIG. 2 illustrates an exemplary block diagram representation of a computer-implemented system, such as those shown in FIG. 1, capable of determining a blood sugar level in a sample obtained from a user, comparing a determined blood sugar level with a predefined blood sugar threshold value, and to analyze the blood sugar level, in accordance with an embodiment of the present disclosure;

FIG. 3 is a flow diagram 300 representing the steps involved in a method for screening pregnant women for gestational diabetes using the organic glucose beverage containing 50-gram and 100-gram serving of the organic glucose beverage of the present invention, in accordance with an embodiment of the present invention; and

FIG. 4 is a flow diagram representing the steps involved in a method for screening pregnant women for gestational diabetes using the organic glucose beverage containing 75-gram serving of the organic glucose beverage of the present invention, in accordance with an embodiment of the present invention.

Further, those skilled in the art will appreciate that elements in the figures are illustrated for simplicity and may not have necessarily been drawn to scale. Furthermore, in terms of the method steps, chemical compounds, and parameters used herein may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more components, compounds, and ingredients preceded by “comprises . . . a” does not, without more constraints, preclude the existence of other components or compounds or ingredients, or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

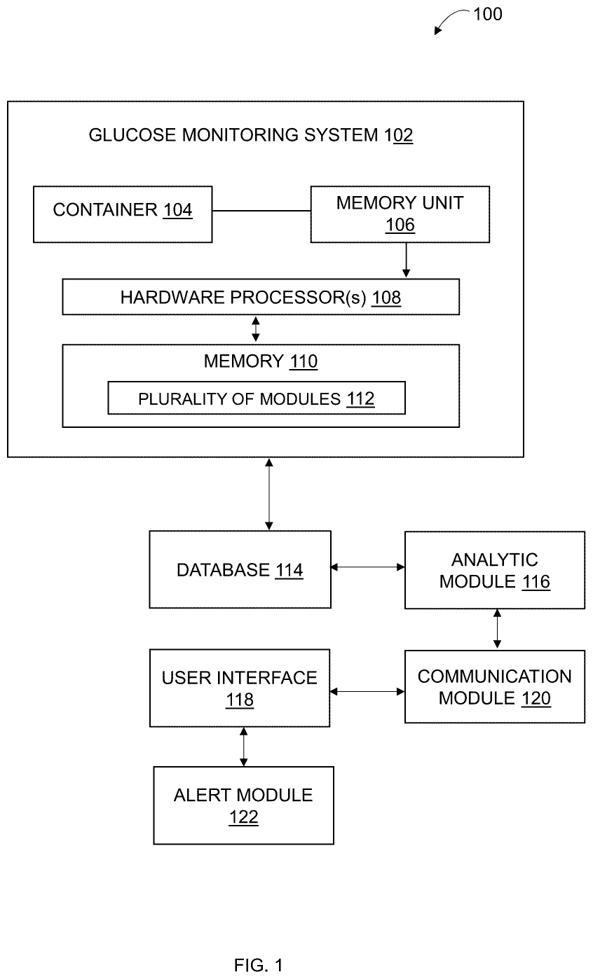

FIG. 1 illustrates an exemplary block diagram 100 representation of a glucose monitoring system 102 for diabetes screening, diagnosis, and treatment of hypoglycemia including gestational diabetes mellitus (GDM), type-1 diabetes, type-2 diabetes, and human and veterinary treatment of hypoglycemia, in accordance with an embodiment of the present disclosure; and

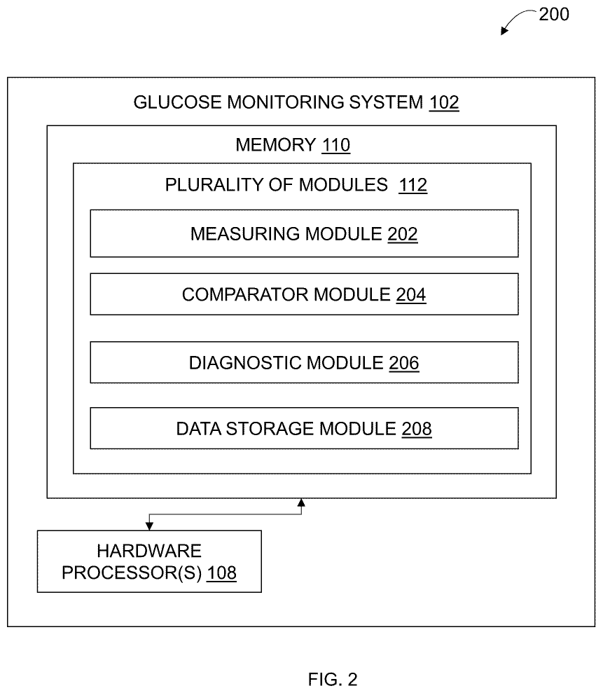

FIG. 2 illustrates an exemplary block diagram 200 representation of a computer-implemented system, such as those shown in FIG. 1, capable of determining a blood sugar level in a sample obtained from a user, comparing a determined blood sugar level with a predefined blood sugar threshold value, and to analyze the blood sugar level, in accordance with an embodiment of the present disclosure.

Embodiments of the present invention relate to an organic glucose beverage and a novel glucose monitoring system that significantly improves a process of diabetes screening, diagnosis, and treatment of hypoglycemia including gestational diabetes mellitus (GDM), type-1 diabetes, type-2 diabetes, and human and veterinary treatment of hypoglycemia. The organic glucose beverage is provided in the form of a ready-to-drink beverage. The organic glucose beverage of the present invention is a certified organic beverage such as U.S. Department of Agriculture (USDA) certified organic beverage that is not only free from toxic additives and artificial compounds but also shelf-stable, eliminating the need for refrigeration, and ensuring both patient safety and convenience.

In an embodiment of the present invention, an organic glucose beverage for diabetes screening, diagnosis, and treatment of hypoglycemia including gestational diabetes mellitus (GDM), type-1 diabetes, type-2 diabetes, and human and veterinary treatment of hypoglycemia is provided. The organic glucose beverage comprises 19-40% w/v organic glucose in the form of dextrose monohydrate, 3.5% w/v juice concentrates; 0.5% w/v citric acid, and balance to 100% w/v water. All ingredients are taken in a liquid form. The organic glucose beverage is provided in the form of the ready-to-drink beverage. The organic glucose beverage is both free from artificial dyes, additives, and toxic preservatives, and shelf-stable for an extended period without refrigeration.

In one exemplary embodiment, the organic glucose beverage is USDA certified organic beverage. The organic glucose beverage is suitable for use in diabetes screening, diagnosis, and treatment of hypoglycemia including gestational diabetes mellitus (GDM), type-1 diabetes, type-2 diabetes, and human and veterinary treatment of hypoglycemia. The organic glucose beverage is further suitable for use in a neonatal intensive care unit (NICU) for treating hypoglycemic newborns, and suitable for use with all ages for hypoglycemia, as well as veterinary uses with animals. The organic glucose beverage is administered to neonates in a dropper form.

In one exemplary embodiment, the organic glucose beverage comprises dosage of 50 to 100 grams. A 10-ounce serving of the organic glucose beverage comprises 50 grams of the organic glucose for screening gestational diabetes mellitus (GDM). A larger serving of the organic glucose beverage comprises 100 grams of the organic glucose for additional diagnostic testing. The organic glucose beverage is provided in pre-measured dosage to ensure accurate glucose consumption for each specific test. The organic glucose beverage is formulated for a gastrointestinal tolerance of a user during glucose testing. The organic glucose beverage is provided in single-use, ready-to-drink containers designed to ensure accurate glucose intake for each test. The organic glucose beverage enables a healthcare provider in screening and diagnosing gestational diabetes mellitus (GDM), type-1 diabetes, and type-2 diabetes. The organic glucose beverage further enables the healthcare provider in treating hypoglycemia in humans and animals. The organic glucose beverage is suitable for use in a neonatal intensive care unit (NICU) for treating hypoglycemic newborns, wherein the organic glucose beverage is free of citric acid.

In one exemplary embodiment, the 50-gram dosage of the organic glucose beverage comprises 19-40% w/v organic glucose in the form of dextrose monohydrate, 3.5% w/v juice concentrates, 0.5% w/v citric acid; and 76% w/v water.

In one exemplary embodiment, the 75-gram dosage of the organic glucose beverage comprises 29-30% w/v organic glucose in the form of dextrose monohydrate, 3.5% w/v juice concentrates, 0.5% w/v citric acid; and 66% w/v water.

In one exemplary embodiment, the 100-gram dosage of the organic glucose beverage comprises 39-40% w/v organic glucose in the form of dextrose monohydrate, 3.5% w/v juice concentrates, 0.5% w/v citric acid; and 56% w/v water.

In another embodiment of the present invention, a glucose monitoring system (102) for diabetes screening, diagnosis, and treatment of hypoglycemia including gestational diabetes mellitus (GDM), type-1 diabetes, type-2 diabetes, and human and veterinary treatment of hypoglycemia is provided. The glucose monitoring system comprises a container 104 holding an organic glucose beverage comprising 19-40% w/v organic glucose in form of dextrose monohydrate, 3.5% w/v juice concentrates; 0.5% w/v citric acid, and balance to 100% w/v water, one or more hardware processors 108, and a memory unit 106 operatively connected to the one or more hardware processors 108, wherein the memory unit 106 comprises a set of computer-readable instructions in form of a plurality of modules 112, configured to be executed by the one or more hardware processors 108. The plurality of modules 112 comprises a measuring module 202 for determining a blood sugar level in a sample obtained from a user. The organic glucose beverage is consumed by the user within a predefined time period prior to a sample collection. A comparator module 204 configured for comparing the determined blood sugar level with a predefined blood sugar threshold value, a diagnostic module 206 configured to analyze the blood sugar level determined by the measuring module. The diagnostic module compares the determined blood sugar level to the predefined blood sugar threshold value and provides a diagnosis of a glycemic status of the user, indicating one of: a presence and absence of diabetes based on the comparison, following the consumption of the organic glucose beverage within the predefined time period, and a data storage module 208 for storing historical data of the blood sugar levels and a user-specific information. The glucose monitoring system 102 is configured to recommend a dosage of the organic glucose beverage to be administered, based on user's medical history and current health condition, while the diagnostic module 206 is configured to diagnose diabetes by analyzing the measured blood sugar levels compared with the predefined blood sugar threshold value. The glucose monitoring system 102 is configured for diagnosing gestational diabetes mellitus (GDM) in pregnant women, type-1 diabetes, and type-2 diabetes. The predefined blood sugar threshold value is based on real-time analysis of blood sugar data of the user, and user-specific parameters consisting of age, weight, and health history. The predefined time for measuring the blood sugar level is between 60 to 180 minutes after consumption of the organic glucose beverage depending on a test performed. The test performed consists one or more of a 50 g test, 75 g test and 100 g test.

In one exemplary embodiment, the one or more hardware processor 108 is configured to execute the computer-readable instructions for receiving the determined blood sugar levels from the diagnostic module 206, comparing the determined blood sugar level to the predefined blood sugar threshold value, analyzing the comparison results using a predefined algorithm, and generating an automated diagnosis of diabetes in real-time based on the analysis.

In another embodiment of the present invention, the glucose monitoring system (102) further comprises a database 114 configured to store test results of the user an analytic module 116 configured to analyze the user's test results, medical history, and current health condition to provide trend analysis over time, facilitating the monitoring of glucose levels during one of pregnancy state and ongoing diabetes management of the user, a user interface 118 configured to enable the healthcare provider to customize thresholds based on various user's demographics, a communication module 120 configured to dynamically transmit at least one of the determined blood sugar levels, the predefined blood sugar threshold value, the comparison results, and generated automated diagnosis of diabetes related to each user to one or more remote database, cloud storage, and one or more device associated with the healthcare provider, and an alert module 122 configured to provide one or more alert signals to at least one of a healthcare provider and the user, if blood sugar levels exceed or fall below the predefined blood sugar threshold value.

In one exemplary embodiment, the user's demographics, as referenced in the user interface 118, encompass a range of characteristics that enable healthcare providers to customize thresholds for blood sugar management effectively. These demographics include age, which can significantly influence physiological responses to glucose; gender, which may affect insulin sensitivity and metabolism; weight and height, which are essential for calculating body mass index (BMI) and assessing overall health; and medical history, including any coexisting conditions that may alter blood sugar levels or treatment efficacy. Additionally, ethnicity is considered due to its association with varying risks for diabetes and related complications. Activity level and dietary habits are also critical factors, as they directly impact glucose metabolism and overall health management. By incorporating these demographic elements, the user interface 118 empowers healthcare providers to tailor thresholds to each individual's unique profile, ensuring more effective and personalized diabetes care. This customization is essential for optimizing treatment strategies, improving patient outcomes, and facilitating proactive health management.

In one exemplary embodiment, the predefined blood sugar threshold value is established through a comprehensive real-time analysis of the user's blood sugar data, integrating user-specific parameters to enhance personalized diabetes management. This process begins by continuously monitoring the user's glucose levels, allowing for immediate detection of fluctuations. The analysis incorporates critical factors such as age, which affects insulin sensitivity and metabolic rates; weight, which is essential for calculating body mass index (BMI) and understanding overall health; and health history, including any existing medical conditions or previous diabetes management strategies that may influence blood sugar regulation. By synthesizing these parameters with real-time data, the system dynamically adjusts the blood sugar threshold value, ensuring that it reflects the user's current health status and needs. This tailored approach enables healthcare providers to implement timely interventions, optimize treatment plans, and improve patient outcomes, thereby facilitating more effective management of diabetes and its associated risks.

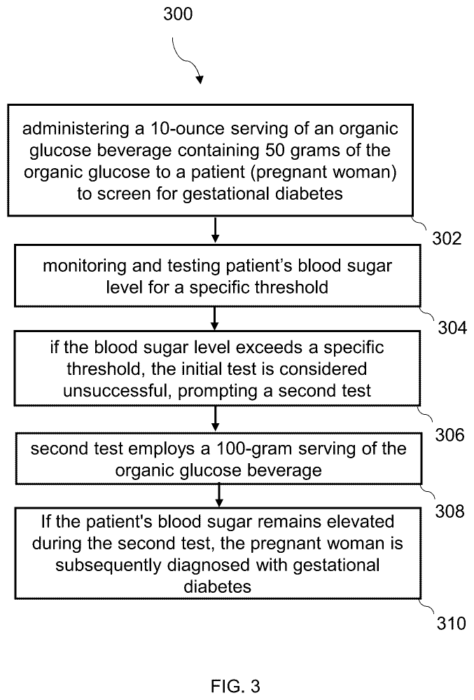

FIG. 3 is a flow diagram 300 representing the steps involved in a method for screening pregnant women for gestational diabetes using the organic glucose beverage containing 50-gram and 100-gram serving of the organic glucose beverage of the present invention, in accordance with an embodiment of the present invention.

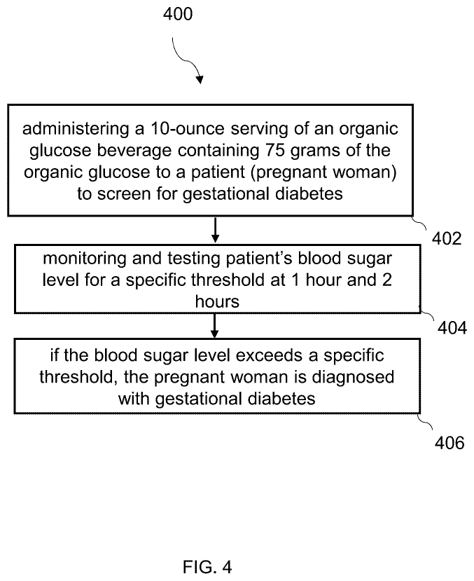

FIG. 4 is a flow diagram 400 representing the steps involved in a method for screening pregnant women for gestational diabetes using the organic glucose beverage containing 75-gram serving of the organic glucose beverage of the present invention, in accordance with an embodiment of the present invention.

In another embodiment of the present invention, a method for screening one or more of gestational diabetes mellitus (GDM), type-1 diabetes, and type-2 diabetes is provided. The method comprises steps of administering a 10-ounce serving of an organic glucose beverage. The organic glucose beverage comprises 19-20% w/v organic glucose in the form of dextrose monohydrate, 3% w/v juice concentrates; 0.5% w/v citric acid, and balance to 100% w/v water, measuring a blood sugar level of a user at a predefined time after consumption of the organic glucose beverage, and determining whether a blood sugar level exceeds a predefined blood sugar threshold value, thereby diagnosing one or more of gestational diabetes mellitus (GDM), type-1 diabetes, and type-2 diabetes. The predefined time for measuring the blood sugar level is between 60 to 180 minutes after consumption of the organic glucose beverage depending on a test performed. The test performed consists one or more of a 50 g test, 75 g test and 100 g test.

The present invention is explained further in the following specific examples, which are only by way of illustration and are not to be construed as limiting the scope of the invention. All testing and specs are created in compliance to the food and drug administration (FDA) and American diabetes association (ADA) standards.

### EXAMPLES

### Example 1: Screening of Pregnant Women for Gestational Diabetes

In obstetrics, a 10-ounce serving of the organic glucose beverage, containing 50 grams of the organic glucose, is administered to a patient (pregnant woman) at approximately 25 weeks of pregnancy to screen for gestational diabetes, at step 302. Following consumption of the organic glucose beverage, the patient's blood sugar level is tested, at step 304. At step 306, if the blood sugar level exceeds a specific threshold, the initial test is considered unsuccessful, prompting a second test. The second test employs a 100-gram serving of the organic glucose beverage, at step 308. At step 310, if the patient's blood sugar remains elevated during the second test, the pregnant woman is subsequently diagnosed with gestational diabetes. The organic glucose beverage is available in 50 g, 75 g, 100 g and as glucose shots for treatment of hypoglycemia and as smaller droppers for NICU/inpatient/veterinary uses.

### Example 2: Organic Glucose Beverage for Diabetes Screening

The “two-step” glucose tolerance test involves a 50 g initial test and a 100 g follow-up test if the 50 g test results are abnormal. The 50 g test serves as a screening test, conducted first. If the individual's blood glucose exceeds a certain threshold during this test, they proceed to the 100 g test. The 100 g test is diagnostic; failing it confirms a diagnosis of diabetes. Those who pass the 50 g test do not need to undergo the 100 g test. Alternatively, the 75 g test, known as a “one-step” test, although less commonly used, can serve for both screening and diagnosing diabetes. A 10-ounce serving of the organic glucose beverage, containing 75 grams of the organic glucose, is administered to a patient (pregnant woman) at approximately 25 weeks of pregnancy to screen for gestational diabetes, at step 402. Following consumption of the organic glucose beverage, the user or patient's blood sugar level is monitored and tested for a specific threshold at 1 hour and 2 hours, at step 404. At step 406, if the blood sugar level exceeds a specific threshold, the pregnant woman is diagnosed with gestational diabetes.

### Example 3: Organic Glucose Beverage for Diabetes Treatment

Low blood sugar is very dangerous and can happen quickly after the dosage of insulin and other diabetes medication use. The glucose beverage quickly increases blood sugar level during hypoglycemia back to healthy levels. Glucose is a sugar that brain and cells recognize and use for energy. It's the most quickly absorbed source of carbohydrates and is used to treat hypoglycemia, especially in people with diabetes. The organic glucose beverage is used in shot or dropper form to quickly increase blood glucose levels in hypoglycemia including one or more of a patient who has taken extra dose of insulin, a diabetic patient who took their diabetes medication then skipped meals, a newborn that is born premature, has feeding issues, is underweight, etc., the user or patient having low blood sugar and needs treatment.

The patient's blood is drawn to check their fasting blood glucose level, then the 10 oz organic glucose beverage is consumed to increase blood sugar levels. If the patient has diabetes (insulin resistance), their blood sugar will increase higher than someone who does not have diabetes. The blood is drawn at 1, 2, or 3 hours depending on the test, and the value of blood glucose levels determines if the patient has insulin resistance or not. The organic beverage is more palatable, has removed all toxic chemicals/toxic preservatives including brominated vegetable oil (BVO), potassium sorbate, sodium benzoate, artificial flavors and colors that are known to cause harm to a health of the user or patients). For the 50 g test, blood is drawn at 1 hour after the organic glucose beverage is consumed. For the 75 g test, blood is drawn at 1 hour and again at 2 hours after the organic glucose beverage is consumed. For the 100 g test, blood is drawn three times: once at 1 hour, 2 hours, and 3 hours after the organic glucose beverage is consumed.

In a healthy patient without diabetes: Fasting blood sugar levels needs to be lower than 95 mg/dL. After one hour, blood sugar levels need to be lower than 180 mg/dL. After two hours, blood sugar levels need to be lower than 155 mg/dL. After three hours, blood sugar levels need to be lower than 140 mg/dL. When the blood sugar is above the thresholds, it is confirmed that the user or patient has diabetes.

The following compositions are examples based on various medical uses as claimed in the patent, ensuring accurate glucose dosages, shelf-stability, and compliance with organic standards.

### Example 4: Gestational Diabetes Screening (50 Grams Glucose per 10-Ounce Serving)

Glucose (Dextrose Monohydrate): 19% w/v (50 grams in 10 ounces, equivalent to ˜300 ml);
Juice Concentrate: 3.5% w/v (9 grams),
   Example: One or more of organic blueberry concentrates or organic lime concentrate for mild sweetness;
Citric Acid: 0.5% w/v (1.5 grams);
Water: 76% w/v.

A 10-ounce (approximately 300 mL) serving contains 57 grams of organic glucose (dextrose monohydrate). This concentration is ideal for screening gestational diabetes, with 50 grams being the effective dosage in a slightly reduced serving size (˜8.77 oz or ˜260 mL). The juice concentrates provide a pleasant flavor, and citric acid ensures product stability and pH control.

### Example 5: Diagnostic Testing (100 Grams Glucose per 16-Ounce Serving)

Organic glucose (dextrose monohydrate): 20% w/v;
Juice concentrates: 3.5% w/v,
   Example: One or more of organic blueberry concentrates or organic lime concentrate for mild sweetness;
Citric acid: 0.5% w/v; and
Water: 56% w/v

In this formulation a 16-ounce (approximately 480 mL) serving contains 96 grams of organic glucose, suitable for diagnostic testing requiring higher glucose levels. The serving size or concentration can be adjusted slightly (e.g., 500 mL) to reach exactly 100 grams of glucose. The increased glucose concentration is balanced with a higher amount of juice concentrate for flavoring and citric acid to ensure pH stability.

### Example 6: Hypoglycemia Treatment for Neonates (Without Citric Acid)

Organic glucose (dextrose monohydrate): 19% w/v;
Juice concentrates (e.g., organic blueberry concentrates or organic lime concentrate): 3.5% w/v;
Water: Balance to 100% w/v;

In this formulation, the beverage is designed for hypoglycemic newborns in a neonatal intensive care unit (NICU). The organic glucose content is moderate to provide a gentle increase in blood sugar without overwhelming the infant's system. No citric acid is included, ensuring the formulation is gentle on newborns and free from any acidity.

### Example 7: Hypoglycemia Treatment for Adults (50 Grams Glucose per 1-3 Ounce Serving)

Organic glucose (dextrose monohydrate): ˜10-15 g;
Juice concentrates (e.g., organic blueberry concentrates or organic lime concentrate): 3.5% w/v;
Citric acid: 0.5% w/v; and
Water: 76% w/v.

The organic glucose beverage is also utilized on occasion for the diagnosis of type 1 diabetes. In this process, a patient consumes the organic glucose beverage, followed by a blood sugar test. If the blood sugar level exceeds a specific threshold, a diagnosis of diabetes is confirmed.

The present invention provides first time an organic beverage for diabetes screening, diagnosis, and treatment of hypoglycemia including gestational diabetes mellitus (GDM), type-1 diabetes, type-2 diabetes, and human and veterinary treatment of hypoglycemia. The organic glucose beverage of the present invention is healthier, well tolerated, and does not include artificial flavors or dyes which sets it apart from conventional glucose solutions for use in diabetes screening and diagnosis.

Therefore, the present invention provides an organic glucose beverage and a novel glucose monitoring system for diabetes screening, diagnosis, and treatment of hypoglycemia including gestational diabetes mellitus (GDM), type-1 diabetes, type-2 diabetes, and human and veterinary treatment of hypoglycemia. The organic glucose beverage eliminates the potential risks associated with synthetic additives, artificial dyes, and toxic preservatives that have been prevalent in conventional glucose solutions. This safety aspect is particularly critical in screening for gestational diabetes mellitus (GDM) among pregnant women and in diagnosing type-1 diabetes, and type-2 diabetes, where patient well-being is paramount.

The ready-to-drink and shelf-stable nature of the organic glucose beverage streamlines the screening process reducing complexities associated with preparation, storage, and refrigeration. This not only simplifies the screening process but also minimizes logistical burdens for healthcare facilities.

The integrated monitoring system in conjunction with the organic glucose beverage of the present invention significantly enhances accuracy in diabetes screening and diagnosis. The system provides precise blood sugar measurements contributing to reliable and dependable results.

Additionally, the composition of the organic glucose beverage in ready-to-drink form may improve patient compliance in cases involving children and pregnant women. The improved patient compliance can lead to more consistent and trustworthy test outcomes.

Furthermore, the present invention offers potential cost savings in healthcare settings. By mitigating expenses related to the preparation, storage, and disposal of traditional glucose solutions containing synthetic components, the present invention provides a cost-effective alternative for healthcare providers. Also, the versatility of the glucose monitoring system is another key advantage in accommodating various diabetes screening scenarios including gestational diabetes mellitus (GDM), type-1 diabetes, and type-2 diabetes. This versatility ensures that the present invention can be a valuable tool for healthcare providers across a spectrum of patient needs and clinical contexts.

Therefore, the organic glucose beverage of the present invention provides numerous advantages including safety, convenience, accuracy, improved compliance, cost-effectiveness, versatility, and a commitment to quality and health-consciousness. With the potential to revolutionize diabetes management, the present invention benefits individuals of all ages and demographics, making it a significant advancement in healthcare.

While specific language has been used to describe the invention, any limitations arising on account of the same are not intended. As would be apparent to a person skilled in the art, various working modifications may be made to the method in order to implement the inventive concept as taught herein.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, order of processes described herein may be changed and are not limited to the manner described herein. Moreover, the actions of any flow diagram need not be implemented in the order shown; nor do all of the acts need to be necessarily performed. Also, those acts that are not dependent on other acts may be performed in parallel with the other acts. The scope of embodiments is by no means limited by these specific examples.

What is claimed is:

1. An organic glucose beverage for diabetes screening, diagnosis, and treatment of hypoglycemia, comprising:
19-40% w/v organic glucose in the form of dextrose monohydrate;
3.5% w/v juice concentrates;
0.5% w/v citric acid; and
balance to 100% w/v water,
wherein the organic glucose beverage is in a ready-to-drink form, free of artificial dyes, toxic preservatives, and additives, and shelf-stable for an extended period without refrigeration.

2. The organic glucose beverage of claim 1, wherein the organic glucose beverage comprises dosage one or more of 50 grams, 75 grams, and 100 grams.

3. The organic glucose beverage of claim 2, wherein the 50-gram dosage of the organic glucose beverage comprises
19-20% w/v organic glucose in the form of dextrose monohydrate;
3.5% w/v juice concentrates;
0.5% w/v citric acid; and
77% w/v water.

4. The organic glucose beverage of claim 2, wherein the 75-gram dosage of the organic glucose beverage comprises
29-30% w/v organic glucose in the form of dextrose monohydrate;
3.5% w/v juice concentrates;
0.5% w/v citric acid; and
66% w/v water.

5. The organic glucose beverage of claim 2, wherein the 100-gram dosage of the organic glucose beverage comprises
39-40% w/v organic glucose in the form of dextrose monohydrate;
3.5% w/v juice concentrates;
0.5% w/v citric acid; and
56% w/v water.

6. The organic glucose beverage of claim 2, wherein a 10-ounce serving of the organic glucose beverage comprises 50 grams of the organic glucose for screening gestational diabetes mellitus (GDM).

7. The organic glucose beverage of claim 2, wherein a larger serving of the organic glucose beverage comprises 100 grams of the organic glucose for additional diagnostic testing.

8. The organic glucose beverage of claim 1, wherein the organic glucose beverage is provided in pre-measured dosage to ensure accurate glucose consumption for each specific test.

9. The organic glucose beverage of claim 1, wherein the organic glucose beverage is formulated for a gastrointestinal tolerance of a user during glucose testing,
the organic glucose beverage is provided in single-use, ready-to-drink containers designed to ensure accurate glucose intake for each test.

10. The organic glucose beverage of claim 1, wherein the organic glucose beverage enables a healthcare provider in screening and diagnosing gestational diabetes mellitus (GDM), type-1 diabetes, and type-2 diabetes.

11. The organic glucose beverage of claim 1, wherein the organic glucose beverage further enables the healthcare provider in treating hypoglycemia in humans and animals.

12. The organic glucose beverage of claim 1, wherein the organic glucose beverage is suitable for use in a neonatal intensive care unit (NICU) for treating hypoglycemic newborns, wherein the organic glucose beverage is free of citric acid.

13. The organic glucose beverage of claim 12, wherein the organic glucose beverage is administered to neonates in a dropper form.

14. A glucose monitoring system for screening and diagnosing diabetes, comprising:
a container holding an organic glucose beverage comprising 19-40% w/v organic glucose in form of dextrose monohydrate, 3.5% w/v juice concentrates; 0.5% w/v citric acid, and balance to 100% w/v water;
one or more hardware processors; and
a memory unit operatively connected to the one or more hardware processors, wherein the memory unit comprises a set of computer-readable instructions in form of a plurality of modules, configured to be executed by the one or more hardware processors, wherein the plurality of modules comprises:
a measuring module configured for determining a blood sugar level in a sample obtained from a user, wherein the organic glucose beverage is consumed by the user within a predefined time period prior to a sample collection;
a comparator module configured for comparing the determined blood sugar level with a predefined blood sugar threshold value,
a diagnostic module configured to analyze the blood sugar level determined by the measuring module, wherein the diagnostic module compares the determined blood sugar level to the predefined blood sugar threshold value and provides a diagnosis of a glycemic status of the user, indicating one of a: presence and absence of diabetes based on the comparison, following the consumption of the organic glucose beverage within the predefined time period; and
a data storage module configured for storing historical data of the blood sugar levels and a user-specific information;
wherein the glucose monitoring system is configured to recommend a dosage of the organic glucose beverage to be administered, based on user's medical history and current health condition, while the diagnostic module is configured to diagnose diabetes by analyzing the measured blood sugar levels compared with the predefined blood sugar threshold value.

15. The glucose monitoring system of claim 14, wherein the glucose monitoring system is configured for diagnosing gestational diabetes mellitus (GDM) in pregnant women, type-1 diabetes, and type-2 diabetes.

16. The glucose monitoring system of claim 14, wherein the one or more hardware processor is configured to execute the computer-readable instructions for:
receiving the determined blood sugar levels from the diagnostic module;
comparing the determined blood sugar level to the predefined blood sugar threshold value;
analyzing the comparison results using a predefined algorithm; and
generating an automated diagnosis of the diabetes in real-time based on the analysis.

17. The glucose monitoring system of claim 14, further comprising:
a database configured to store test results of the user;
an analytic module configured to analyze the test results, medical history, and current health condition of the user to provide trend analysis over time, facilitating the monitoring of glucose levels during one of pregnancy state and ongoing diabetes management of the user;
a user interface configured to enable the healthcare provider to customize thresholds based on various user's demographics; and
a communication module configured to dynamically transmit at least one of the determined blood sugar levels, the predefined blood sugar threshold value, the comparison results, and generated automated diagnosis of diabetes related to each user to one or more remote database, cloud storage, and one or more device associated with the healthcare provider; and
an alert module configured to provide one or more alert signals to at least one of a healthcare provider and the user, if a blood sugar level exceeds or fall below the predefined blood sugar threshold value.

18. The glucose monitoring system of claim 14, wherein the predefined blood sugar threshold value is based on real-time analysis of blood sugar data of the user, and user-specific parameters consisting of age, weight, and health history.

19. A method for screening one or more of gestational diabetes mellitus (GDM), type-1 diabetes, and type-2 diabetes, comprising:
administering a 10-ounce serving of an organic glucose beverage, wherein the organic glucose beverage comprises 19-40% w/v organic glucose in the form of dextrose monohydrate, 3.5% w/v juice concentrates; 0.5% w/v citric acid, and balance to 100% w/v water;
measuring a blood sugar level of a user at a predefined time after consumption of the organic glucose beverage; and
determining whether a blood sugar level exceeds a predefined blood sugar threshold value, thereby diagnosing one or more of gestational diabetes mellitus (GDM), type-1 diabetes, and type-2 diabetes.

20. The method of claim 19, wherein the predefined time for measuring the blood sugar level is between 60 to 180 minutes after consumption of the organic glucose beverage depending on a test performed,
the test performed consists one or more of a 50 g test, 75 g test and 100 g test.
